(19) United States
(12) Patent Application Publication
(10) Pub. No.: US 2008/0177320 A1
(43) Pub. Date: Jul. 24, 2008

(54) Vertebral Rods and Methods of Use

(75) Inventor: Larry Thomas McBride, Memphis, TN (US)

Correspondence Address: COATS & BENNETT/MEDTRONICS, 1400 CRESCENT GREEN, SUITE 300, CARY, NC, 27518, US

(73) Assignee: Warsaw Orthopedic, Inc., Warsaw, IN (US)

(21) Appl. No.: 11/554,074

(22) Filed: Oct. 30, 2006

(51) Int. Cl. A61B 17/56

(52) U.S. Cl. 606/261

(57) ABSTRACT

The present application is directed to vertebral rods constructed for vertebral movement in first and second planes, and to prevent or inhibit vertebral movement in a third plane. The vertebral rod may include one or more notches. The notches change the cross section shape of the rod and thus the structural characteristics. The notches may be shaped, sized, and positioned to facilitate vertebral movement in the first and second planes, and prevent or inhibit movement in the third plane. A fill material may be positioned within the notches to strengthen the rod and/or provide durability.

### BACKGROUND

Spinal or vertebral rods are often used in the surgical treatment of spinal disorders such as degenerative disc disease, disc herniations, scoliosis or other curvature abnormalities, and fractures. Different types of surgical treatments are used. In some cases, spinal fusion is indicated to inhibit relative motion between vertebral bodies. In other cases, dynamic implants are used to preserve motion between vertebral bodies. For either type of surgical treatment, spinal rods may be attached to the exterior of two or more vertebrae, whether it is at a posterior, anterior, or lateral side of the vertebrae. In other embodiments, spinal rods are attached to the vertebrae without the use of dynamic implants or spinal fusion.

Spinal rods may provide a stable, rigid column that encourages bones to fuse after spinal-fusion surgery. Further, the rods may redirect stresses over a wider area away from a damaged or defective region. Also, a rod may restore the spine to its proper alignment. In some cases, a flexible rod may be appropriate. Flexible rods may provide some advantages over rigid rods, such as increasing loading on interbody constructs, decreasing stress transfer to adjacent vertebral elements while bone-graft healing takes place, and generally balancing strength with flexibility.

Aside from each of these characteristic features, a surgeon may wish to control anatomic motion after surgery. That is, a surgeon may wish to inhibit or limit one type of spinal motion while allowing a lesser or greater degree of motion in a second direction. As an illustrative example, a surgeon may wish to inhibit or limit motion of lateral bending while allowing a greater degree of flexion and extension. However, conventional rods tend to be symmetric in nature and may not provide this degree of control.

### SUMMARY

The present application is directed to vertebral rods that support one or more vertebral members. The rod may include one or more notches that alter the structural characteristics. The rods provide for vertebral movement in first and second planes, and prevent or inhibit vertebral movement in a third plane. Fill material may be positioned within the notches to support the rod as it bends during vertebral movement. In one embodiment, the rod provides for flexion, extension and rotational movement while limiting or preventing lateral bending.

### BRIEF DESCRIPTION OF THE DRAWINGS

FIG. 1 is a perspective view of a device according to one embodiment.

FIG. 2 is schematic coronal view of a device attached to a scoliotic spine according to one embodiment.

FIG. 3 is sectional view taken along line III-III of FIG. 1.

FIG. 4 is sectional view of a device according to one embodiment.

FIG. 5 is side view of a device according to one embodiment.

FIG. 6 is a perspective view of a device according to one embodiment.

FIG. 7 is a sectional view of a device according to one embodiment.

FIG. 8 is a perspective view of a device according to one embodiment.

FIG. 9 is a perspective view of a device according to one embodiment.

FIG. 10 is a side view of a device according to one embodiment.

FIG. 11 is a perspective view of a device according to one embodiment.

### DETAILED DESCRIPTION

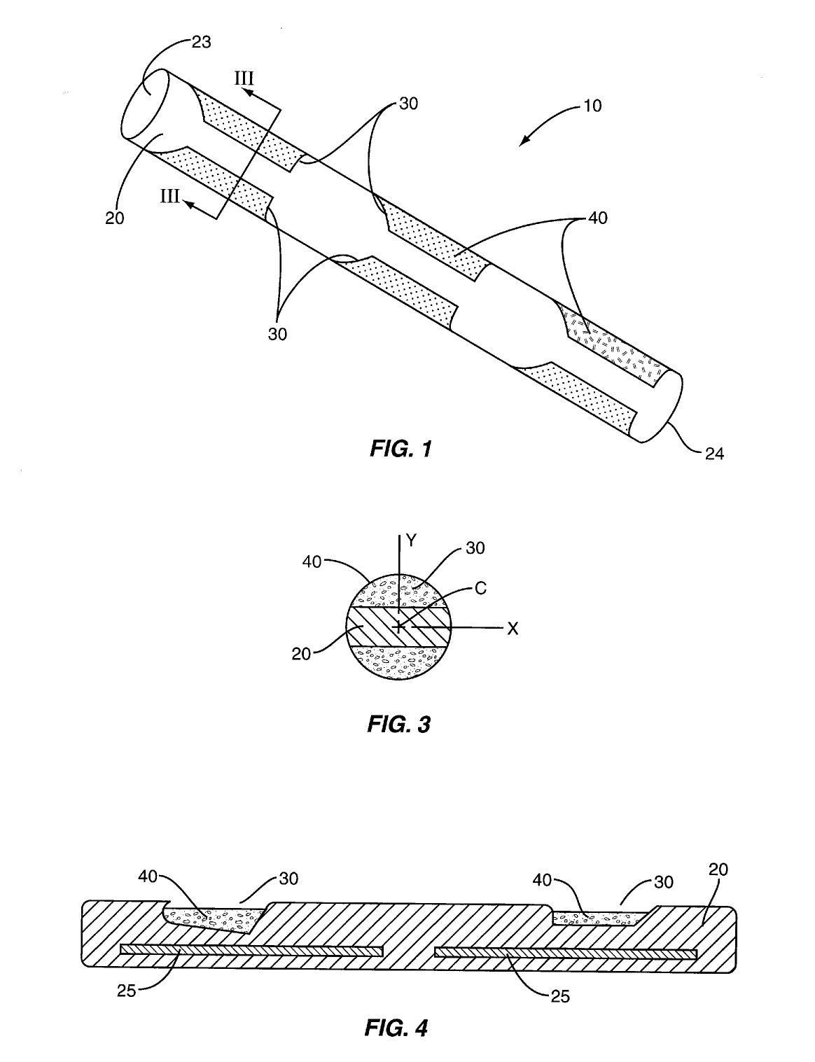

The present application is directed to vertebral rods constructed for vertebral movement in first and second planes, and to prevent or inhibit vertebral movement in a third plane. FIG. 1 illustrates one embodiment of a device 10 that includes a rod 20 sized to extend along one or more vertebral members. One or more notches 30 are positioned within the rod 20. The notches 30 alter the structural characteristics of the rod 20 to provide for specific motion of the vertebral members. Fill material 40 is positioned within the notches 30 to support the rod 20 as it bends during vertebral movement.

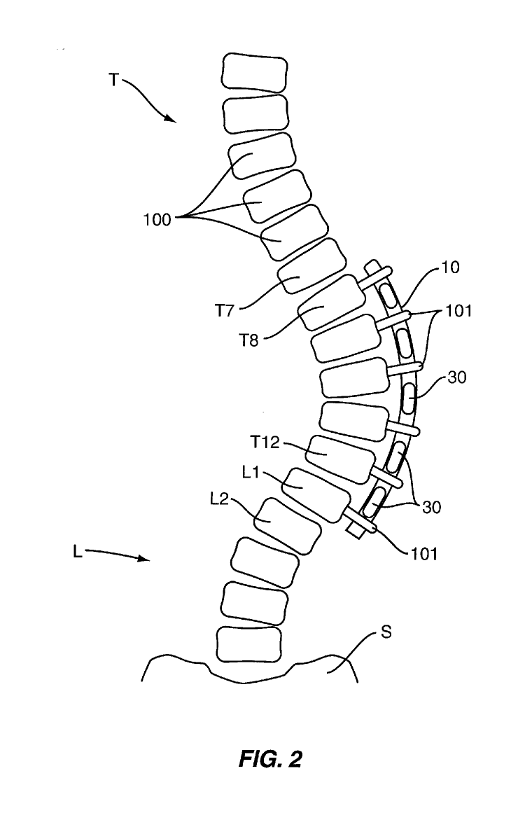

FIG. 2 illustrates a patient's spine that includes the vertebral members 100 of the thoracic region T, the lumbar region L, and the sacrum S. This spine has a scoliotic curve with an apex of the curve being offset from its correct alignment in the coronal plane. The spine is deformed laterally so that the axes of the vertebral members 100 are displaced from the sagittal plane passing through a centerline of the patient. The device 10 is attached to vertebral members 100 with one or more fasteners 101. The device 10 allows flexion, extension, and axial rotation with two planes while limiting lateral bending in a third plane. These constraints on motion maintain kyphosis, lordosis, and coronal balance while controlling the scoliotic deformity.

Returning to FIG. 1, rod 20 includes an elongated shape with first and second ends 23, 24. When not under the influence of any exterior forces, rod 20 may be substantially straight or may be curved. Rod 20 may include a variety of cross-sectional shapes including but not limited to substantially circular as illustrated in FIGS. 1 and 3, oval, substantially rectangular as illustrated in FIG. 6, or a combination such as illustrated in FIG. 7. Rod 20 may be solid along the entire length, or hollow along a section or entirety of the length.

Rod 20 may further include one or more support members 25 as illustrated in FIG. 4. Support members 25 are elongated members positioned within the rod 20 for further strength and support. FIG. 4 illustrates one embodiment with the support members 25 axially spaced along the length. In another embodiment illustrated in FIG. 7, multiple support members 25 are positioned in an overlapping arrangement. Support members 25 may be constructed of a variety of materials, and may include a variety of lengths and cross-sectional shapes. In embodiments with multiple support members 25, the members 25 may be constructed of the same or different materials.

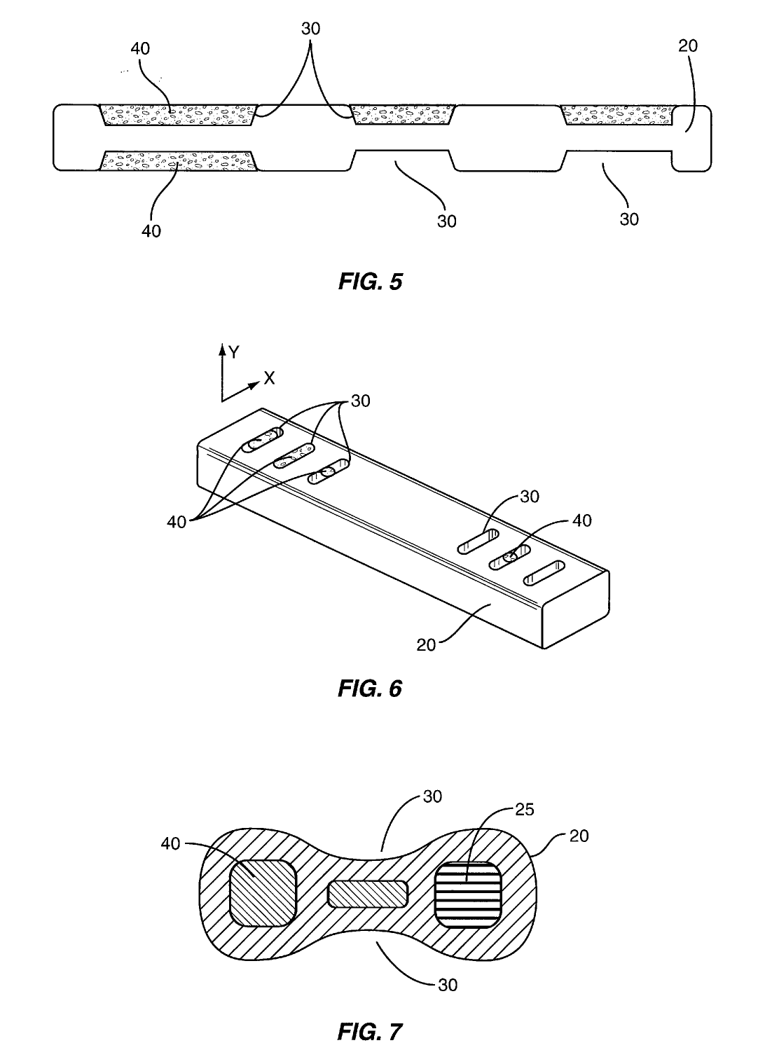

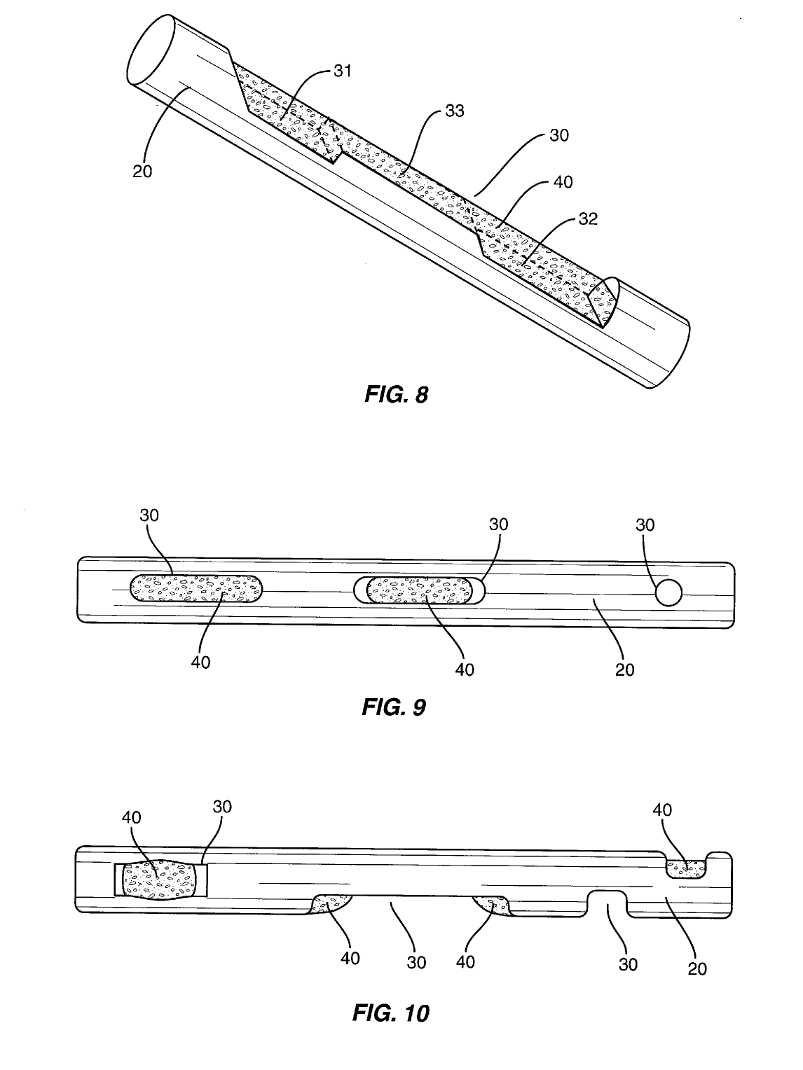

One or more notches 30 extend into the rod 20. Notches 30 may include a symmetrical shape as illustrated in FIG. 5. Notches 30 may also be asymmetrical as illustrated in FIG. 8 with different depths and surface configurations at different sections. In the embodiment of FIG. 8, notch 30 includes a first section 31 with a first depth, a second section 32 with a second, different depth, and an intermediate section 33 with yet another different depth.

In some embodiments, notches 30 are positioned on the exterior of the rod 20 as illustrated in FIGS. 1, 5, and 8. An exterior notch 30 is not bounded on opposing sides by the rod 20. Notches 30 may also extend through an interior of the rod 20 as illustrated in FIGS. 6, 9, and 10. Interior notches 30 extend through an interior of the rod 20 and are bounded on opposing sides by the rod 20.

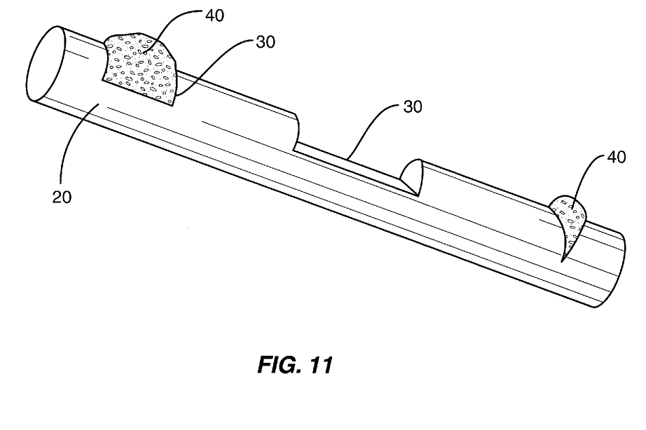

In one embodiment as illustrated in FIG. 8, a single notch 30 extends into the rod 20. In other embodiments, multiple notches 30 extend into the rod 20. In one embodiment as illustrated in FIG. 1, notches 30 extend into the rod 20 from multiple sides. In one specific embodiment as illustrated in FIGS. 1 and 5, the notches 30 extend inward from opposing sides. In another embodiment as illustrated in FIG. 10, notches 30 are positioned in a staggered pattern such that there is no overlap of notches 30 along the length. In yet another embodiment, multiple notches 30 are positioned with some overlap among the notches 30. Other combinations are possible, including for example, embodiments with sections of the length including some overlap of the notches 30 and other sections of the length with no overlap of the notches 30. FIG. 11 illustrates another embodiment with multiple notches 30 each extending from substantially the same side of the rod 20.

The rod 20 may be constructed from a variety of surgical grade materials. These include metals such as stainless steels, cobalt-chrome, titanium, and shape memory alloys. Non-metallic rods, including polymer rods made from materials such as PEEK and UHMWPE, are also contemplated.

The structural characteristics of the rod 20 and notches 30 provide vertebral bending in one or more directions, and prevent or limit bending in a another direction. Using the example of FIG. 2, movement is provided within the sagittal plane and prevented or limited within the coronal plane. The structural characteristics may be dependent upon several factors, including the material choice of the rod 20, and the cross section shape. The flexural rigidity, which is a measure of bending stiffness, is given by the equation:

Flexural Rigidity=E×I (1)

where E is the modulus of elasticity or Young's Modulus for the rod material and I is the moment of inertia of a rod cross section about the bending axis. The modulus of elasticity varies by material and reflects the relationship between stress and strain for that material. As an illustrative example, titanium alloys generally possess a modulus of elasticity in the range between about 100-120 GPa. By way of comparison, implantable grade polyetheretherketone (PEEK) possesses a modulus of elasticity in the range between about 3-4 Gpa, which, incidentally, is close to that of cortical bone.

In general, an object's moment of inertia depends on its shape and the distribution of mass within that shape. The greater the concentration of material away from the object's centroid C, the larger the moment of inertia. The centroid C may be the center of mass for the shape assuming the material is uniform over the cross section. FIG. 3 illustrates a cross section of the notched area of the rod 20 of FIG. 1. Since the width of the cross section area in the direction of the x axis is larger than the width in the direction of the y axis, it follows that the moment of inertia in the x-axis Iₓ is larger than the moment of inertia in the y-axis I_{y}. This means that there is a greater resistance to bending in the x axis as compared to the y-axis. That is, the device 10 will bend about the x axis (up-and-down as illustrated in FIG. 3) easier than it will bend about the y axis (left-and-right). Again using the embodiment of FIG. 2, the rod 20 may be positioned with the x-axis substantially parallel to the coronal plane to prevent lateral bending and allow for flexion and extension. The surgeon may also elect to install the rod 10a with the x and y axes oriented at angles other than aligned with the sagittal and coronal planes of the patient.

Outside of the notch 30 regions, the rod 20 of FIG. 3 is substantially symmetrical and therefore does not include structural characteristics that would facilitate bending in one or more planes and prevent of eliminate bending in another plane. Therefore, the positioning, shape, and size of the notches 30 cause the structural characteristics that control the bending. In other embodiments, the structural characteristics are caused by a combination of the rod shape and notches 30.

FIG. 6 illustrates a rod 20 with a substantially rectangular cross section. A major axis extends along the x-axis and a minor axis along the y-axis. This shape results with the moment of inertia in the x-axis Iₓ being larger than the moment of inertia in the y-axis I_{y}. This results with a greater resistance to bending in the x axis as compared to the y-axis. The interior notches 30 that extend through the rod 20 lessen the resistance to bending in the x-axis. This may facilitate bending the rod 20 to conform to the curvature of the spine during initial placement into the patient.

Another manner of affecting the ability to bend is the placement of one or more support members 25 within the rod 20. The flexural rigidity of the members 25 determined by the modulus of elasticity and the moment of inertia of a member cross section may be used to further adjust the overall structural characteristics of the device 10.

One example of a vertebral rod with various bending stiffness is disclosed in U.S. patent application Ser. No. 11/342,195 entitled “Spinal Rods Having Different Flexural Rigidities about Different Axes and Methods of Use”, filed on Jan. 27, 2006, hereby incorporated by reference.

Fill material 40 is positioned within the notches 30 to strengthen the rod 20 and/or provide durability. The fill material 40 includes a modulus of elasticity or Young's Modulus that is less than the rod 20. Therefore, the strength and durability of the rod 20 with the fill material 40 is less than a non-notched rod 20. Fill material 40 may include a variety of different substances, including but not limited to carbon fiber, polycarbonates, silicone, polyetheretherketone, and combinations thereof.

Varying amounts of fill material 40 may be positioned within the notches 30. In embodiments as illustrated in FIGS. 1 and 5, fill material 40 substantially fills the notches 30. In another embodiment as illustrated in FIGS. 4 and 10, fill material 40 fills less than the entirety of the notches 30. In still other embodiments, fill material 40 fills and extends outward from the notches 30 as illustrated in FIG. 11. Multiple notch embodiments may also include variations in the amount of fill material 40 in the various notches 30. In some multiple notch embodiments, one or more of the notches may not include fill material 40.

In one embodiment, during vertebral motion in a first direction, the body 20 is bent and one or more of the notches 30 are deformed and decreased in size. This deformation also causes fill material within these notches 30 to be deformed.

The devices and methods may be used to treat spinal deformities in the coronal plane, such as a scoliotic spine illustrated in FIG. 2. The devices and methods may also be used to treat deformities in the sagittal plane, such as a kyphotic spine or Scheurmann's kyphosis. The devices may also be used to provide support to damaged vertebral members 100 and intervertebral discs that have been damaged from various causes including a specific event such as trauma, a degenerative condition, a tumor, or infection.

In one embodiment, the device 10 is inserted into the patient in a percutaneous manner. The device 10 may be deformed into a shape that mirrors the spine's curvature. One embodiment includes accessing the spine from an anterior approach to the cervical spine. Other applications contemplate other approaches, including posterior, postero-lateral, antero-lateral and lateral approaches to the spine, and accessing other regions of the spine, including the cervical, thoracic, lumbar and/or sacral portions of the spine.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A vertebral rod comprising:
an elongated body constructed of a first material;
a plurality of notches spaced along the body, the plurality of notches causing the body to bend within first and second planes and substantially prevent bending within a third plane; and
a fill material positioned within each of the plurality of notches, the fill material being different than the first material.

2. The rod of claim 1, wherein the body further includes non-notched sections including a symmetrical cross-sectional shape.

3. The rod of claim 1, wherein the body further includes non-notched sections including a non-symmetrical cross-sectional shape.

4. The rod of claim 1, further including at least one support member extending within the body, the at least one support member constructed of a third material that is different than the body and the fill material.

5. The rod of claim 1, wherein the plurality of notches are positioned in an overlapping arrangement.

6. The rod of claim 1, wherein the plurality of notches are positioned on a common side of the body.

7. The rod of claim 1, wherein the fill material extends outward from at least one of the plurality of notches.

8. The rod of claim 1, wherein at least one of the notches is an interior notch that extends through the body.

9. A vertebral rod comprising:
an elongated body constructed of a first material;
a notch extending into the body; and
a fill material positioned within the notch;
the body, notch, and fill material cause a first flexural rigidity for bending in a first direction and a second flexural rigidity to substantially prevent bending in a second direction.

10. The rod of claim 9, wherein a modulus of elasticity for the body and the fill material is different.

11. The rod of claim 9, wherein the notch includes different sections each including different depths.

12. The rod of claim 9, wherein the first and second directions are about 90° apart.

13. A vertebral rod comprising:
an elongated body constructed of a first material;
a notch extending into the body; and
a fill material positioned within each of the plurality of notches, the fill material being different than the first material;
the body including a first cross section shape away from the notch and a second cross section shape at the notch, the shapes causing the body to bend within first and second planes and substantially prevent bending within a third plane.

14. A vertebral rod comprising:
an elongated body constructed of a first material;
a notch extending into the body and causing the body to bend within first and second planes and substantially prevent bending within a third plane; and
a fill material positioned within the notch, the fill material being different than the first material to strengthen the body during bending within the first and second planes.

15. The rod of claim 14, wherein the body includes a symmetrical shape away from the notch and an asymmetrical shape at the notch.

16. The rod of claim 15, wherein the body includes a substantially circular cross section shape.

17. The rod of claim 14, wherein the body includes a substantially rectangular cross section shape.

18. The rod of claim 14, further comprising a support member extending within the body to strengthen the body, the support member constructed of a different material than the body and the fill material.

19. The rod of claim 14, further comprising a second notch extending into the body, the second notch being spaced away from the notch.

20. The rod of claim 14, wherein the fill material extends outward from the notch.

21. A vertebral rod comprising:
an elongated body constructed of a first material;
a first notch and a second notch each extending into the body and causing the body to bend within first and second planes and substantially prevent bending within a third plane;
a fill material positioned within the first and second notches, the fill material being different than the first material to strengthen the body during bending within the first and second planes; and
a support member extending along the body to support the body, the support member being constructed of a different material than the body and the fill material.

22. The rod of claim 21, therein the support member is positioned in overlapping arrangement with one of the first and second notches and the fill material.

23. A method of supporting vertebral members with a vertebral rod, the method comprising the steps of:
bending an elongated body during vertebral motion in a first direction;
decreasing a size of the notch and deforming a fill material within the notch during the vertebral motion in the first direction; and
maintaining the size of the notch and preventing deformation of the fill material to inhibit vertebral motion in a second direction.

24. The method of claim 23, further comprising decreasing a second notch and deforming a second fill material within the second notch during the vertebral motion in the first direction.
